(19) United States
(12) Patent Application Publication
(10) Pub. No.: US 2004/0171696 A1
(43) Pub. Date: Sep. 2, 2004

(54) Pharmaceutical composition for the treatment of the decline and/or damage of cognitive functions

(76) Inventors: Istvan Gacsalyi, Budapest (HU); Gyorgy Levay, Budakeszi (HU); Ildiko Nagyne Gyonos, Budapest (HU); Laszlo Gabor Harsing, Budapest (HU)

Correspondence Address: BIRCH STEWART KOLASCH & BIRCH, PO BOX 747, FALLS CHURCH, VA, 22040-0747, US

(21) Appl. No.: 10/483,808

(22) PCT Filed: Jul. 18, 2002
(86) PCT No.: PCT/HU02/00069

(30) Foreign Application Priority Data
Jul. 18, 2001 (HU) P 01 03017

(51) Int. Cl. A61K 31/137

(52) U.S. Cl. 514/650

(57) ABSTRACT

The compounds of the general Formula (I) (wherein R stands for hydrogen or methyl) can be used for the treatment or prophylaxis of diseases characterized either by the decline and/or damage of cognitive functions, or mental disability accompanying other diseases. As compound of the general Formula I preferably -(1R,2S,4R)-(−)-2-(2-dimethylaminoethoxy)-2-phenyl-1,7,7-trimethylbicyclo[2.2.1]heptane-fumarate can be used.

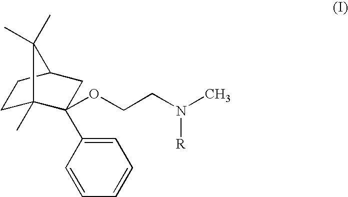

### FIELD OF THE INVENTION

The present invention relates to a pharmaceutical composition for treatment or prophylaxis of diseases characterized by either the decline and/or damage of cognitive functions, or mental impairment accompanying other diseases, particularly to a pharmaceutical composition for the treatment of Parkinson's disease, Korsakoff syndrome or Huntington's disease.

### TECHNICAL BACKGROUND

It is known that 1,7,7-trimethylbicyclo[2.2.1]heptane derivatives bearing a phenyl, phenyl-alkyl, or thienyl group and a dialkylaminoalkyl side chain in position 2 display anticonvulsive, motor-depressant, analgetic effect, and potentiate hexobarbital-induced narcosis (GB Patent No. 2 065 122). One of the prominent representatives of this compound group is (1R,2S,4R)-(−)-2-(2-dimethylaminoethoxy)-2-phenyl-1,7,7-trimethylbicyclo[2.2.1]heptane, known as deramciclane, in form of free base and its pharmaceutically acceptable acid addition salts, particularly the fumarate salt. Such compounds are disclosed in Hungarian Patent No. 212 547.

Deramciclane displayed significant effect on the animal models of anxiety and depression. In the Vogel lick-conflict model, deramciclane was effective after 1 and 10 mg/kg per os treatment in rats. In the social interaction model [File S.E.J. Neurosci. Methods, 2, 219-238 (1980)], deramciclane increased the number of social interactions following as low as 0.7 mg/kg intraperitoneal treatment. In the light-dark model [Crawley J. N., Pharmacol. Biochem. and Behaviour, 15, 695-699 (1981)], deramciclane showed anxiolytic activity following 3 mg/kg single subcutaneous administration. In the marble burying test considered to be experimental model of obsessive-compulsive disorder [Broekkamp C. L. et al, Eur. J. Pharmacol. 126, 223-229, (1986)], deramciclane displayed significant activity following per os administration of 10 and 30 mg/kg doses.

In the elevated plus-maze, deramciclane alone was completely inactive, however, antagonized CCK agonist induced anxiety [Gacsályi et al, Drug Dev. Res. 40, 333-348 (1997)].

The anxiolytic effects summarized above are accompanied by antidepressant effects as well. In the learned helplessness considered to be the experimental model of depression [Grial et al, Biol. Psychiatry, 23, 237-242 (1988)] deramciclane was found to be effective after intraperitoneal administration of 1 and 10 mg/kg doses.

As for receptor profile, deramciclane basically binds to central 5-HT_{2C} and 5-HT_{2A} receptors. This unique receptor profile might explain the above summarized anxiolytic and antidepressant effects as well [Gacsályi et al, Drug Dev. Res. 40, 333-348 (1997)].

The present invention is directed to a pharmaceutical composition for the treatment or prophylaxis of diseases characterized either by the decline and/or damage of cognitive functions, or mental disability accompanying other diseases.

Basically, two types of memory functions are known to exist according to the literature. In the first type, known as short term memory, the learned information is stored for a period between some minutes and a few hours. In the second type, known as long term memory, the information may be kept from hours to years [Baddley and Warington, J. Verb. Learn. Verb. Behav. 9, 176-179 (1970)]; Wright et al, Science 229, 287-289 (1985)].

The process of information being transferred from short term memory to long term memory is called memory consolidation.

The process of recalling or manifesting information from either short term or long term memory is called memory retention. Total amnesia is relatively rare, however, diseases accompanied by various levels of memory impairment occur in a continuously growing number. At present, approximately 18 million patients are suffering from Alzheimer's disease and this number will be doubled in the next 25 years for this disease only [Fletcher, Mol. Med. Today, 3/10 p. 429-434, (1997)].

### ESSENCE OF THE INVENTION

It is the object of the present invention to develop a new pharmaceutical composition being effective for treating diseases or conditions accompanied by various degrees of memory dysfunction.

The above object is achieved by means of the present invention.

The present invention is based on the recognition that the bicycloheptane derivatives of general Formula

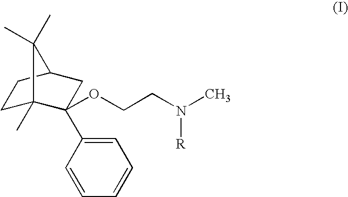

(wherein R stands for hydrogen or methyl) can be efficiently used for the treatment or prophylaxis of diseases characterized either by the decline and/or damage of cognitive functions, or mental disability accompanying other diseases.

Within the above indications, the compounds of general Formula I can be advantageously used for treating or preventing Parkinson's disease, Korsakoff syndrome or Huntington's disease, particularly mental disability consequent on stroke, mental disability consequent on other CNS catastrophes, Alzheimer's disease, dementia, in particular senile dementia of the elderly, etc.

According to the present invention there is provided a process for the preparation of a pharmaceutical composition comprising a bicycloheptane derivative of the general Formula I (wherein R stands for hydrogen or methyl) or a pharmaceutically acceptable acid addition salt thereof which comprises admixing the active ingredient prepared in a known manner with pharmaceutical carriers and/or diluents and finishing in the form of pharmaceutical compositions for the treatment or prophylaxis of diseases characterized either by the decline and/or damage of cognitive functions, or mental disability accompanying other diseases.

### DETAILS OF THE INVENTION

Within the context of the present invention the general Formula I encompasses all optical isomers and mixtures thereof.

As compound of the general Formula I preferably (1R,2S,4R)-(−)-2-(2-dimethylaminoethoxy)-2-phenyl-1,7,7-trimethylbicyclo[2.2.1]heptane or pharmaceutically acceptable acid addition salts thereof can be used.

The term “pharmaceutically acceptable acid addition salt” relates to salts formed with pharmaceutically acceptable inorganic acids (e.g. hydrogen halides, such as hydrochloric acid or hydrogen bromide; sulfuric acid, nitric acid or phosphoric acid etc.) or organic acids (e.g. tartaric acid, succinic acid, malic acid, lactic acid, citric acid, maleic acid, fumaric acid, methanesulfonic acid, p-toluene-sulfonic acid etc.). According to a particularly preferred embodiment of the present invention a fumarate is used.

According to the most preferable embodiment of the present invention (1R,2S,4R)-(−)-2-(2-dimethylaminoethoxy)-2-phenyl-1,7,7-trimethyl-bicyclo[2.2.1]heptane-fumarate is used.

According to a further aspect of the present invention there is provided a pharmaceutical composition for the treatment or prophylaxis of diseases characterized either by the decline and/or damage of cognitive functions, or mental disability accompanying other diseases comprising as active ingredient a compound of the general Formula I (wherein R stands for hydrogen or methyl) or a pharmaceutically acceptable acid addition salt thereof in admixture with suitable inert solid or liquid pharmaceutical carriers and/or auxiliary agents.

The daily dosage of the compounds of the general Formula I depends on the circumstances of the given case (e.g. body weight, age and condition of the patient, the disease to be treated, the severity of the damage etc.). The daily dose of the compound of the general Formula I is generally 0.01-10 mg/kg, preferably 0.1-1.0 mg/kg.

The pharmaceutical compositions according to the present invention can be prepared by methods of pharmaceutical industry known per se.

For oral administration tablets, dragées, enterosolvent tablets or dragées or hard or soft gelatine capsules can be used. The active ingredient content of such compositions may be 10-100 mg per dosage unit. The above pharmaceutical compositions can contain carriers (preferably silicic acid), binders (e.g. polyvinylpyrrolidone or gelatine), sliding agents, lubricants (e.g. magnesium stearate, talc or sodium lauryl sulfate). Oral aqueous suspensions and/or elixirs can be prepared by admixing the active ingredient with taste-improving agents, dyes, emulsifiers and diluents (e.g. water, ethanol, propylenglycol, glycerol etc.).

Tablets can be prepared by dry or wet granulating methods. In course of the preparation of dragées the dragée core is coated with a suitable coating layer. Capsules are prepared by filling the mixture into hard or soft gelatine capsules.

According to a further aspect of the present invention there is provided the use of compounds of the general Formula I (wherein R stands for hydrogen or methyl) for the treatment or prophylaxis of diseases characterized either by the decline and/or damage of cognitive functions, or mental disability accompanying other diseases.

According to a still further feature of the present invention there is provided a method of treatment or prophylaxis of diseases characterized either by the decline and/or damage of cognitive functions, or mental disability accompanying other diseases which comprises administering to the patient in need of such treatment a pharmaceutically active amount of a compound of the general Formula I or a pharmaceutically acceptable acid addition salt thereof.

Further details of the present invention are to be found in the following Examples without limiting the scope of protection to said Examples.

### EXAMPLE 1

### Determination of Efficacy Displayed by the Compounds of General Formula I for Treating or Preventing Diseases Characterized Either by the Decline and/or Damage of Cognitive Functions or Mental Disability Accompanying Other Diseases

(1R,2S,4R)-(−)-2-(2-dimethylaminoethoxy)-2-phenyl-1,7,7-trimethyl-bicyclo[2.2.1]heptane-fumarate (deramciclane) was employed as test substance.

Method

Male Wistar rats weighing 200-220 g were used. The animals were obtained from Charles River Co. They were kept in a room with normal 12-12 h light dark cycle (light on: 06:00) at a relative humidity of 60±10%.

The experiment was performed in a five-channel “step through”-type passive avoidance learning apparatus. The equipment consisted of two adjacent Plexi-glass boxes of 20×20×16 cm. One of them was made of regular transparent Plexi-glass and the other one was made of black, non-transparent Plexi-glass. The boxes were connected with a 7.5×8 cm passage way, equipped with a computer-controlled guillotine-door. The passage of the rats through the door was detected by infrared photocells arranged in two parallel lines in the opening of the passage way. The door was automatically closed when the animals passed through. The dark compartment was equipped with stainless steel grid floor through which electric foot shocks could be delivered to the animals. A 10 W light bulb was installed above the passage way in the light compartment.

The experiment was performed on two consecutive days, in two sessions which were 24 h apart from each other.

On Day 1 (Acquisition) the animals acquired information about the situation (grid floor shock in the dark compartment), on Day 2 (Retention) they recalled the acquired information to avoid punishment (“if I go into the dark I will be punished, so I stay outside in the light”).

Day 1 (Acquisition)

The individually numbered animals were placed into the light compartment of the equipment. After 30 s the guillotine door was opened and the rats could freely pass to the dark (considered as safe) compartment. Step through latency was automatically determined. (Step through latency is the time period spanning from door opening to the time when the animal passed into the dark compartment.) The door was closed then, and the timer was automatically stopped. An electric foot shock of 1.2 mA lasting 2.5 s was applied to the animal through the grid floor 3 s after the door has been closed, except for rats in the absolute control group (no shock+vehicle treated). Test animals were removed from the dark compartment immediately after foot shock has been delivered. The function of the absolute control group was to shown that shocked animals will remember to the unpleasant foot shock as revealed by increased latency time when compared to absolute control. That is the essence of acquisition.

Day 2 (Retention)

After 24 h the animals were placed again in the light compartment of the test apparatus and step-through latency was measured as described at Acquisition day, except that no foot shock was applied to the animals in any group on the second day. A maximum of 180 s time interval was available for the rats to pass into the dark compartment. The animals were removed from the light compartment if they did not pass to the dark compartment within the 180 s test period.

Treatments

For examining the impact on learning the animals were treated intraperitoneally in a volume of 1 ml/kg either with (1R,2S,4R)-(−)-2-(2-dimethylaminoethoxy)-2-phenyl-1,7,7-trimethylbicyclo[2.2.1]heptane-fumarate or with vehicle (0.4% methyl-cellulose) on Day 1, 30 min before Acquisition. When effect on retention from long term memory was investigated the animals were treated intraperitoneally in a volume of 1 ml/kg on Day 2, 30 min before testing Retention. Data were analyzed using two way ANOVA, followed by Duncan's post hoc test for between group differences.

Results and Discussion

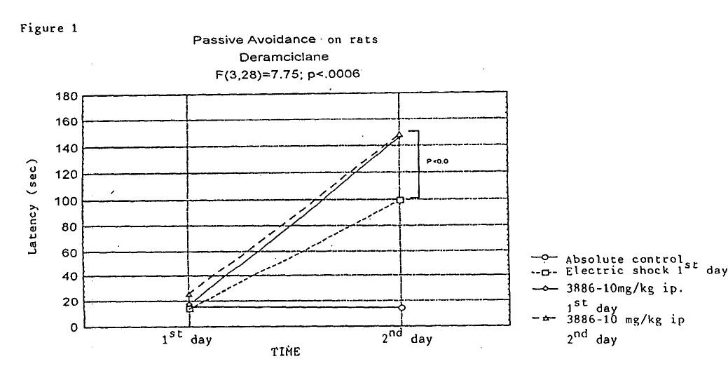

It has been surprisingly found that (1R,2S,4R)-(−)-2-(2-dimethylaminoethoxy)-2-phenyl-1,7,7-trimethyl-bicyclo[2.2.1]heptane-fumarate, known for its anxiolytic effect as major CNS effect, significantly increased step-through latency into the dark compartment of the passive avoidance apparatus both after Day 1 and/or Day 2 administration of the compound (FIG. 1).

It is shown on FIG. 1 that in absolute control group (no shock, untreated), step-through latency was approximately the same on both experimental days (this means that there was nothing to recall and avoid on the second day).

In the shocked, vehicle treated control group the unavoidable 1.2 mA foot shock resulted in a significantly increased step-through latency on Day 2 when compared to absolute control. The experimental animals recalled the annoying experience (foot shock) in the dark, therefore, they passed into the dark compartment after a significantly longer time (increased latency).

In the treated groups this augmented latency was further increased following both types of treatment. After Day 1 treatment, (probably) the acquisition of the information improved significantly, while after Day 2 treatment the retention of memory improved.

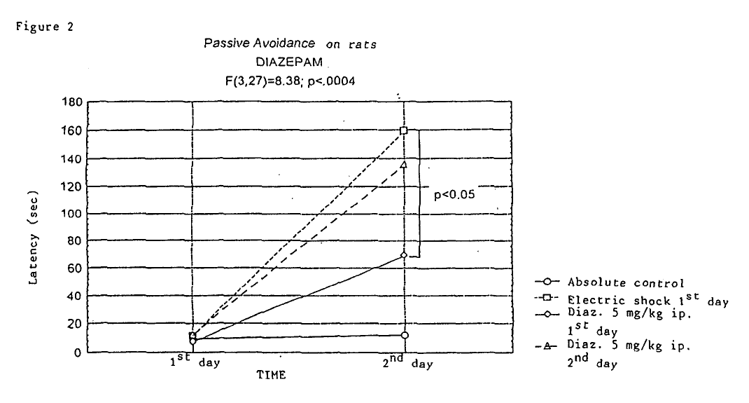

The above recognition is so much the more surprising as anxiolytic compounds either do not influence memory (i.e. buspirone) or have a deleterious effect thereon (i.e. diazepam, FIG. 2).

According to our results, 5 mg/kg ip diazepam administered on Day 1 significantly damaged retention from long term memory as shown on FIG. 2.

From the therapeutic point of view the advantageous effect of (1R,2S,4R)-(−)-2-(2-dimethylaminoethoxy)-2-phenyl-1,7,7-trimethylbicyclo[2.2.1]heptane-fumarate on learning and memory signifies that the compound could be appropriate for treating and/or preventing diseases or conditions accompanying diseases wherein learning or memory functions are suffering a loss or there is a possibility to suffering a loss. Such diseases are—as mentioned above, but not limited to—Alzheimer's disease, Korsakoff syndrome, Huntington's disease and mental decline due to aging processes or impairment of the cognitive functions due to exposure to toxic substances as well.

### EXAMPLE 2

Tablets having the following compositions are prepared by methods of pharmaceutical industry known per se.

| | Amount |
|---|---|
| Component | mg/tablet |
| (1R,2S,4R)-(-)-2-(2-dimethylaminoethoxy)-2- | 20 |
| phenyl-1,7,7-trimethylbicyclo[2.2.1]heptane- | |
| fumarate | |
| Maize starch | 90 |
| Polyvinyl pyrrolidone | 68 |
| Magnesium stearate | 2 |
| Total weight | 180 |

### EXAMPLE 3

Gelatine capsules having the following compositions are prepared by methods of pharmaceutical industry known per se.

| | Amount |
|---|---|
| Component | mg/capsule |
| (1R,2S,4R)-(-)-2-(2-dimethylaminoethoxy)-2- | 20 |
| phenyl-1,7,7-trimethylbicyclo[2.2.1]heptane- | |
| fumarate | |
| Maize starch | 212 |
| Aerosil ® | 5 |
| Magnesium stearate | 3 |
| Total weight | 240 |

What we claim is:

1. Process for the preparation of a pharmaceutical composition comprising a bicycloheptane derivative of the general Formula 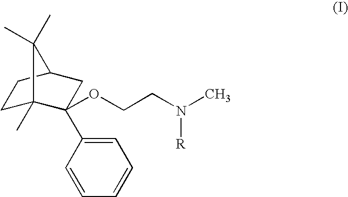
(wherein R stands for hydrogen or methyl) or a pharmaceutically acceptable acid addition salt thereof which comprises admixing the active ingredient prepared in a known manner with pharmaceutical carriers and/or diluents and finishing in the form of pharmaceutical compositions for the treatment or prophylaxis of diseases characterized either by the decline and/or damage of cognitive functions, or mental disability accompanying other diseases.

2. Process according to claim 1 which comprises preparing a pharmaceutical composition for the treatment or prophylaxis of Parkinson's disease, Korsakoff syndrome or Huntington's disease.

3. Process according to claim 2 which comprises preparing a pharmaceutical composition for the treatment or prophylaxis of mental disability consequent on stroke, mental disability consequent on other CNS catastrophes, Alzheimer's disease, dementia, in particular senile dementia of the elderly.

4. Process according to any of claims 1-3 which comprises using (1R,2S,4R)-(−)-2-(2-dimethylamino-ethoxy)-2-phenyl-1,7,7-trimethyl-bicyclo[2.2.1]heptane or a pharmaceutically acceptable acid addition salt thereof as active ingredient.

5. Process according to claim 3 which comprises using (1R,2S,4R)-(−)-2-(2-dimethylamino-ethoxy)-2-phenyl-1,7,7-trimethyl-bicyclo[2.2.1]heptane-fumarate.

6. Process according to any of claims 1-5 which comprises using the compound of the general Formula I or a pharmaceutically acceptable acid addition salt thereof in a daily dose or 0.01-10 mg/kg.

7. Process according to claim 6 which comprises using the compound of the general Formula I or pharmaceutically acceptable acid addition salts thereof in a daily dose of 0.1-1.0 mg/kg.

8. Pharmaceutical composition for the treatment or prophylaxis of diseases characterized either by the decline and/or damage of cognitive functions, or mental disability accompanying other diseases comprising as active ingredient a compound of the general Formula I (wherein R stands for hydrogen or methyl) or a pharmaceutically acceptable acid addition salt thereof in admixture with suitable inert solid or liquid pharmaceutical carriers and/or auxiliary agents.

9. Pharmaceutical composition according to claim 8 for the treatment or prophylaxis of Parkinson's disease, Korsakoff syndrome or Huntington's disease.

10. Pharmaceutical composition according to claim 8 for the treatment or prophylaxis of mental disability consequent on stroke, mental disability consequent on other CNS catastrophes, Alzheimer's disease, dementia, in particular senile dementia of the elderly.

11. Pharmaceutical composition according to any of claims 8-10 comprising (1R,2S,4R)-(−)-2-(2-dimethylaminoethoxy)-2-phenyl-1,7,7-trimethyl-bicyclo[2.2.1]heptane or a pharmaceutically acceptable acid addition salt thereof as active ingredient.

12. Pharmaceutical composition according to claim 11 comprising (1R,2S,4R)-(−)-2-(2-dimethylaminoethoxy)-2-phenyl-1,7,7-trimethyl-bicyclo[2.2.1]heptane-fumarate.

13. Use of compounds of the general Formula I (wherein R stands for hydrogen or methyl) for the treatment or prophylaxis of diseases characterized either by the decline and/or damage of cognitive functions, or mental disability accompanying other diseases.

14. Use according to claim 13 for the treatment or prophylaxis of Parkinson's disease, Korsakoff syndrome or Huntington's disease.

15. Use according to claim 13 for the treatment or prophylaxis of mental disability consequent on stroke, mental disability consequent on other CNS catastrophes, Alzheimer's disease, dementia, in particular senile dementia of the elderly.

16. Use of (1R,2S,4R)-(−)-2-(2-dimethylaminoethoxy)-2-phenyl-1,7,7-trimethylbicyclo[2.2.1]heptane or pharmaceutically acceptable acid addition salts thereof according to claim 13 for the treatment or prophylaxis of diseases characterized either by the decline and/or damage of cognitive functions, or mental disability accompanying other diseases.

17. Use of (1R,2S,4R)-(−)-2-(2-dimethylaminoethoxy)-2-phenyl-1,7,7-trimethylbicyclo[2.2.1]heptane-fumarate according to claim 13 for the treatment or prophylaxis of diseases characterized either by the decline and/or damage of cognitive functions, or mental disability accompanying other diseases.

18. Method of treatment or prophylaxis of diseases characterized either by the decline and/or damage of cognitive functions, or mental disability accompanying other diseases which comprises administering to the patient in need of such treatment a pharmaceutically active amount of a compound of the general Formula I or a pharmaceutically acceptable acid addition salt thereof.

19. Method according to claim 18 for the treatment or prophylaxis of Parkinson's disease, Korsakoff syndrome or Huntington's disease which comprises administering to the patient in need of such treatment a pharmaceutically active amount of a compound of the general Formula I or a pharmaceutically acceptable acid addition salt thereof.

20. Method according to claim 18 for the treatment or prophylaxis of mental disability consequent on stroke, mental disability consequent on other CNS catastrophes, Alzheimer's disease, dementia, in particular senile dementia of the elderly which comprises administering to the patient in need of such treatment a pharmaceutically active amount of a compound of the general Formula I or a pharmaceutically acceptable acid addition salt thereof.

21. Method according to claim 18 which comprises administering to the patient in need of such treatment a pharmaceutically active amount of (1R,2S,4R)-(−)-2-(2-dimethylaminoethoxy)-2-phenyl-1,7,7-trimethyl-bicyclo[2.2.1]heptane or a pharmaceutically acceptable acid addition salt thereof.

22. Method according to claim 21 which comprises administering to the patient in need of such treatment a pharmaceutically active amount of (1R,2S,4R)-(−)-2-(2-dimethylaminoethoxy)-2-phenyl-1,7,7-trimethyl-bicyclo[2.2.1]heptane-fumarate.

23. Method according to any of claims 18-22 which comprises administering the active ingredient in a daily dose of 0.01-10 mg/kg.

24. Method according to claim 23 which comprises administering the active ingredient in a daily dose of 0.1-1.0 mg/kg.
